(19) United States
(12) Patent Application Publication
(10) Pub. No.: US 2023/0288432 A1
(43) Pub. Date: Sep. 14, 2023

(54) TDP-43 BIOSENSOR CELL LINES

(71) Applicant: THE BOARD OF REGENTS OF THE UNIVERSITY OF TEXAS SYSTEM, Austin, TX (US)

(72) Inventors: Marc I. DIAMOND, Austin, TX (US); Jaime VAQUER-ALICEA, Austin, TX (US); Gianna C. MAGGIORE, Austin, TX (US)

(73) Assignee: THE BOARD OF REGENTS OF THE UNIVERSITY OF TEXAS SYSTEM, Austin, TX (US)

(21) Appl. No.: 18/014,850

(22) PCT Filed: Jul. 2, 2021
(86) PCT No.: PCT/US2021/040238

§ 371 Date: Jan. 6, 2023

Related U.S. Application Data
(60) Provisional application No. 63/048,405, filed on Jul. 6, 2020.

(51) Int. Cl. G01N 33/68; C12N 5/071; C12N 15/86; G01N 33/542

(52) U.S. Cl. CPC G01N 33/6896; C12N 5/0686; C12N 15/86; G01N 33/542; G01N 2800/2814; C12N 2503/00; C12N 2740/15043; G01N 2333/4709; G01N 2333/4704

(57) ABSTRACT

The present disclosure provides methods for the identification, characterization and ranking of putative tau monomer stabilizing agents. Specifically, the disclosure provides methods for assessing the capability of a test compound to stabilize a tau monomer, methods of prioritizing a plurality of test compounds from a library identified as tau monomer stabilizing agents, methods of screening a test compound library to identify tau monomer stabilizing agents, and a kit providing the reagents to perform the described methods.

### PRIORITY

This application claims the benefit of U.S. Ser. No. 63/048,405, filed on Jul. 6, 2020, which is incorporated herein by reference in its entirety.

### FIELD OF THE INVENTION

The present disclosure relates to methods for the measurement or detection of TDP-43, the detection of TDP-43-related diseases or disorders, and the identification of TDP-43 prion aggregation inhibitors.

### BACKGROUND

The pathological accumulation of TDP-43 aggregates represents a risk for the development of neurodegenerative diseases; which are hardly detectable, and for which there is few to no existing treatments. Therefore, there is a need for methods for detecting TDP-43, detecting TDP-43-related diseases or disorders, and identifying of TDP-43 prion aggregation inhibitors.

### SUMMARY

Provided herein are expression cassettes, vectors, host cells comprising expression cassettes or vectors, and methods of measuring a titer of or detecting a TDP-43 peptide or aggregate in a sample, methods of detecting amyotrophic lateral sclerosis (ALS), frontotemporal dementia (FTD), or a neuropathological disease or condition linked to TDP-43 in a subject, and methods of identifying a TDP-43 prion aggregation inhibitor.

An embodiment provides an expression cassette comprising one or more polynucleotides encoding a polypeptide at least 95% identical to a sequence as set forth in SEQ ID NO:4 or 8.

The one or more polynucleotides can comprise a sequence at least 95% identical to a sequence as set forth in SEQ ID NO:3 or SEQ ID NO:7. The expression cassette can further comprise a polynucleotide encoding a promoter and a polynucleotide encoding a fluorescent protein. The expression cassette can further comprise a polynucleotide encoding a linker sequence. The linker sequence can be present between the polynucleotide encoding the fluorescent protein and the polynucleotide encoding a polypeptide at least 95% identical to a sequence as set forth in SEQ NO:4 or SEQ ID NO:8. The fluorescent protein can be a fluorescent donor protein or a fluorescent acceptor protein of a proximity fluorescent detection protein pair.

Another embodiment provides a host cell comprising an expression cassette comprising one or more polynucleotides encoding a polypeptide at least 95% identical to a sequence as set forth in SEQ ID NO:4 or 8.

An additional embodiment provides a vector comprising an expression cassette comprising one or more polynucleotides encoding a polypeptide comprise a sequence at least 95% identical to a sequence as set forth in SEQ ID NO:4 or 8. The vector can comprise a polynucleotide at least 95% identical to a sequence as set forth in SEQ ID NO:1, SEQ ID NO:2, SEQ ID NO:5, or SEQ ID NO:6.

An embodiment provides a host cell comprising a first vector comprising a polynucleotide encoding a first TDP-43 polypeptide fragment and a polynucleotide encoding a fluorescent donor protein, and a second vector comprising a polynucleotide encoding a second TDP-43 polypeptide fragment and a polynucleotide encoding a fluorescent acceptor protein; or a vector comprising a first polynucleotide encoding a first TDP-43 polypeptide fragment and a polynucleotide encoding a fluorescent donor protein, and a second polynucleotide encoding a second TDP-43 polypeptide fragment and a polynucleotide encoding a fluorescent acceptor protein. The first polynucleotide can be operably linked to the second polynucleotide.

The first TDP-43 polypeptide fragment and the second TDP-43 polypeptide fragment can be identical TDP-43 polypeptide fragments, or TDP-43 polypeptide fragments of different lengths that can co-assemble. The polynucleotide encoding the first TDP-43 polypeptide fragment can comprise a sequence at least 95% identical to a sequence as set forth in SEQ ID NO:3 and the polynucleotide encoding the second TDP-43 polypeptide fragment can comprise a sequence at least 95% identical to a sequence as set forth in SEQ ID NO:3. The polynucleotide encoding the first TDP-43 polypeptide fragment can comprise a sequence at least 95% identical to a sequence as set forth in SEQ ID NO:7 and the polynucleotide encoding the second TDP-43 polypeptide fragment can comprise a sequence at least 95% identical to a sequence as set forth in SEQ ID NO:7. The fluorescent donor protein and the fluorescent acceptor protein can be members of a proximity detection protein pair. The proximity detection protein pair can be mClover3/mRuby3, EBFP2/mEGFP, ECFP/EYFP, CeruleanNenus, MiCy/mKO, CyPet/YPet, EGFP/mCherry, Venus/mCherry, Venus/tdTomato, or Venus/m Plum. The first vector can comprise a polynucleotide comprising a sequence at least 95% identical to a sequence as set forth in SEQ ID NO:1 and the second vector can comprise a polynucleotide comprising a sequence at least 95% identical to a sequence as set forth in SEQ ID NO:2. The first vector can comprise a polynucleotide comprising a sequence at least 95% identical to a sequence as set forth in SEQ ID NO:5 and the second vector can comprise a polynucleotide comprising a sequence at least 95% identical to a sequence as set forth in SEQ ID NO:6.

Another embodiment provides a method of measuring a titer of or of detecting a TDP-43 peptide or aggregate in a sample comprising: contacting the sample with a host cell comprising a first vector comprising a polynucleotide encoding a first TDP-43 polypeptide fragment and a polynucleotide encoding a fluorescent donor protein, and a second vector comprising a polynucleotide encoding a second TDP-43 polypeptide fragment and a polynucleotide encoding a fluorescent acceptor protein; or a vector comprising a first polynucleotide encoding a first TDP-43 polypeptide fragment and a polynucleotide encoding a fluorescent donor protein, and a second polynucleotide encoding a second TDP-43 polypeptide fragment and a polynucleotide encoding a fluorescent acceptor protein. The first polynucleotide can be operably linked to the second polynucleotide. The host cell can be exposed to an excitation light. An emission light signal can be detected, thereby detecting a TDP-43 peptide or aggregate in the sample.

An additional embodiment provides a method of detecting amyotrophic lateral sclerosis (ALS), frontotemporal dementia (FTD), or a neuropathological disease or condition linked to TDP-43 in a subject comprising: contacting a sample with a host cell comprising a first vector comprising a polynucleotide encoding a first TDP-43 polypeptide fragment and a polynucleotide encoding a fluorescent donor protein, and a second vector comprising a polynucleotide encoding a second TDP-43 polypeptide fragment and a polynucleotide encoding a fluorescent acceptor protein; or a vector comprising a first polynucleotide encoding a first TDP-43 polypeptide fragment and a polynucleotide encoding a fluorescent donor protein, and a second polynucleotide encoding a second TDP-43 polypeptide fragment and a polynucleotide encoding a fluorescent acceptor protein. The first polynucleotide can be operably linked to the second polynucleotide. The host cell can be exposed to an excitation light. The emission light signal can be detected, thereby detecting TDP-43 peptide in the sample.

A sample can be a biological fluid, a tissue sample, or an aggregated material amplified in vitro therefrom. Detecting an emission light signal can indicate that the sample does not comprise TDP-43 peptide or aggregate. A lack of an emission light signal can indicate that the sample comprises TDP-43 peptide or aggregate.

An embodiment provides a method of identifying a TDP-43 prion aggregation inhibitor comprising: contacting a host cell with a putative TDP-43 prion aggregation inhibitor. The host cell can comprise a first vector comprising a polynucleotide encoding a first TDP-43 polypeptide fragment and a polynucleotide encoding a fluorescent donor protein, and a second vector comprising a polynucleotide encoding a second TDP-43 polypeptide fragment and a polynucleotide encoding a fluorescent acceptor protein; or a vector comprising a first polynucleotide encoding a first TDP-43 polypeptide fragment and a polynucleotide encoding a fluorescent donor protein, and a second polynucleotide encoding a second TDP-43 polypeptide fragment and a polynucleotide encoding a fluorescent acceptor protein. The first polynucleotide can be operably linked to the second polynucleotide. The host cell can be exposed to an excitation light. An emission light signal can be detected. A TDP-43 prion aggregation inhibitor can be identified, where a TDP-43 prion aggregation inhibitor can interact with TDP-43 peptide. Detecting an emission light signal can indicate that the putative TDP-43 prion aggregation inhibitor does not inhibit TDP-43 peptide aggregation. A lack of an emission light signal can indicate that the putative TDP-43 prion aggregation inhibitor inhibits TDP-43 peptide aggregation.

A TDP-43 peptide can be a pathological TDP-43 prion. Detecting an emission light signal can be by immunofluorescent microscopy or by flow cytometry.

Provided herein are methods of measuring a titer of or detecting a TDP-43 peptide or aggregate in a sample, methods of detecting amyotrophic lateral sclerosis (ALS), frontotemporal dementia (FTD), or a neuropathological disease or condition linked to TDP-43 in a subject, and methods of identifying a TDP-43 prion aggregation inhibitor; and expression cassettes, vectors, and hosts cells, for practicing the methods described herein.

### BRIEF DESCRIPTION OF THE DRAWINGS

The features, objects and advantages other than those set forth above will become more readily apparent when consideration is given to the detailed description below. Such detailed description makes reference to the following drawings, wherein:

FIG. 1 shows a schematic of 14 different TDP-43 constructs.

FIG. 2 is a graph illustrating the quantification by flow cytometry of 262-414 FRET signal 72 hours after sample addition.

FIG. 3 illustrates representative fluorescent images of 262-414 TDP-43 aggregates after addition of 3 ug of homogenized brain.

FIG. 4 is a graph illustrating the quantification by flow cytometry of 274-414 FRET signal 72 hours after sample addition.

FIG. 5 illustrates representative fluorescent images of 274-414 TDP-43 aggregates after addition of 3 ug of homogenized brain.

### DETAILED DESCRIPTION

Likewise, many modifications and other embodiments of the genetically modified microorganisms and methods described herein will come to mind to one of skill in the art having the benefit of the teachings presented in the foregoing descriptions and the associated drawings. Therefore, it is to be understood that the methods and compositions are not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art.

### Overview

TAR DNA-binding protein 43 (transactive response DNA binding protein 43 kDa, TDP-43, or TDP43) is a protein encoded in human by the TARDBP gene. TDP-43 is 414 amino acid residues long (SEQ ID NO:28) and consists of 4 domains: an N-terminal domain spanning residues 1-76 (NTD) with a well-defined fold that has been shown to form a dimer or oligomer; 2 highly conserved folded RNA recognition motifs spanning residues 106-176 (RRM1) and 191-259 (RRM2), respectively, required to bind target RNA and DNA; an unstructured C-terminal domain encompassing residues 274-414 (CTD), which contains a glycine-rich region, is involved in protein-protein interactions, and harbors most of the mutations associated with familial amyotrophic lateral sclerosis. The full-length protein is a dimer resulting from the self-interaction between two NTD domains. Dimerization of TDP-43 can be propagated to form higher-order oligomers.

Hyper-phosphorylated, ubiquitinated and cleaved form of TDP-43—known as pathologic TDP-43, TDP-43 fragment, or TDP-43 prion—is a major disease-causing protein in ubiquitin-positive, tau-, and alpha-synuclein-negative frontotemporal dementia (FTLD-TDP) and in amyotrophic lateral sclerosis (ALS). Elevated levels of the TDP-43 protein have also been identified in individuals diagnosed with chronic traumatic encephalopathy, and has also been associated with ALS leading to the inference that athletes who have experienced multiple concussions and other types of head injury are at an increased risk for both encephalopathy and motor neuron disease (ALS). Abnormalities of TDP-43 also occur in an important subset of Alzheimer's disease patients, correlating with clinical and neuropathologic features indexes. Misfolded TDP-43 can also be found in the brains of older adults over age 85 with limbic-predominant age-related TDP-43 encephalopathy, (LATE), a form of dementia. Mutations in the TARDBP gene are associated with neurodegenerative disorders including frontotemporal lobar degeneration and amyotrophic lateral sclerosis (ALS). In particular, TDP-43 mutants M337V and Q331K are studied for their roles in ALS.

There is increasing evidence that the accumulation and spread of protein aggregates in neurodegenerative diseases, such as Alzheimer's Disease and Parkinson's Disease occurs via prion mechanisms. According to this model, a natively folded protein undergoes a conformational change and becomes capable of forming pathogenic aggregates. These aggregates then act as templates for self-replication as they spread from cell to cell. Ultimately, this process leads to cellular dysfunction and neurodegeneration. TDP-43 is a protein that forms aggregates and causes neurodegeneration in a range of neurodegenerative diseases, including the majority of cases of amyotrophic lateral sclerosis (ALS) and a substantial fraction of patients with frontotemporal dementia (FTD). Dominant mutations that cause these disorders (and combinations of each) indicate that TDP-43 aggregation causes neurodegeneration. Accordingly, assays to measure the titer of TDP-43 aggregates in human brain or samples prepared in vitro can be useful for diagnosis and drug discovery.

As used herein, diseases and conditions that can be referred to as “TDP-43-related diseases or conditions” can be characterized by the pathological accumulation of TDP-43 aggregates, which is responsible for neurodegeneration. Non-limiting examples of TDP-43-related disease or condition can include frontotemporal dementia (FTLD), amyotrophic lateral sclerosis (ALS), chronic traumatic encephalopathy, Alzheimer's disease (AD), limbic-predominant age-related (LATE), and TDP-43 encephalopathy.

It has not previously been possible to detect functional TDP-43 prions in vitro, and there is no available assays to measure the titer of TDP-43 aggregates in human brain or samples prepared in vitro; however, the methods described herein enable the detection and quantification of pathological forms of TDP-43 in brain tissues, and therefore can be used to diagnose patients more accurately through the analysis of biofluids or tissue samples.

Provided herein are TDP-43 polypeptide fragments, which, when fused to appropriate fluorescent proteins and expressed in host cell lines, enable the detection of pathological forms of TDP-43 in brain tissues. These biosensor cell lines, constitute tools for clinical diagnosis and for drug discovery.

Indeed, using the biosensor cells, the methods described herein allow for the rapid detection of TDP-43 prions, and therefore can also be used to assist in the discovery of novel drugs that can bind pathogenic TDP-43, or that can interfere with its replication in cells.

Therefore, the methods described herein allow for the detection of TDP-43, the detection of TDP-43-related diseases or disorders, and the identification of TDP-43 prion aggregation inhibitors.

Polynucleotides

Polynucleotides refer to nucleic acid molecules comprising deoxyribonucleic acid (DNA) or ribonucleic acid (RNA). Nucleic acid molecules include but are not limited to genomic DNA, cDNA, mRNA, iRNA, miRNA, tRNA, ncRNA, rRNA, and recombinantly produced and chemically synthesized molecules such as aptamers, plasmids, antisense DNA strands, shRNA, ribozymes, nucleic acids conjugated, oligonucleotides or combinations thereof. Polynucleotides can be present as a single-stranded or double-stranded and linear or covalently circularly closed molecule.

Polynucleotides can be obtained from nucleic acid molecules present in, for example, a mammalian cell. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides can be isolated. An isolated polynucleotide can be a naturally-occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid molecules naturally found immediately flanking the recombinant DNA molecule in a naturally-occurring genome is removed or absent. Isolated polynucleotides also include non-naturally occurring nucleic acid molecules. Polynucleotides can encode full-length polypeptides, polypeptide fragments, and variant or fusion polypeptides. “Isolated polynucleotides” can be (i) amplified in vitro, for example via polymerase chain reaction (PCR), (ii) produced recombinantly by cloning, (iii) purified, for example, by cleavage and separation by gel electrophoresis, (iv) synthesized, for example, by chemical synthesis, or (vi) extracted from a sample.

A polynucleotide can comprise, for example, a gene, open reading frame, non-coding region, or regulatory element. A gene is any polynucleotide molecule that encodes a polypeptide, protein, or fragment thereof, optionally including one or more regulatory elements preceding (5′ non-coding sequences) and following (3′ non-coding sequences) the coding sequence. In one embodiment, a gene does not include regulatory elements preceding and following the coding sequence. A native or wild-type gene refers to a gene as found in nature, optionally with its own regulatory elements preceding and following the coding sequence. A chimeric or recombinant gene refers to any gene that is not a native or wild-type gene, optionally comprising regulatory elements preceding and following the coding sequence, wherein the coding sequences and/or the regulatory elements, in whole or in part, are not found together in nature. Thus, a chimeric gene or recombinant gene comprise regulatory elements and coding sequences that are derived from different sources, or regulatory elements and coding sequences that are derived from the same source, but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide. Polynucleotides can encode the polypeptides described herein (e.g., any TDP-43 polypeptide fragment, or variants thereof suitable for the use described herein).

Degenerate polynucleotide sequences encoding polypeptides described herein, as well as homologous nucleotide sequences that are at least about 80, or about 85, 90, 91, 92, 93, 94, 95, 96, 97, 98, or 99% identical to polynucleotides described herein and the complements thereof are also polynucleotides. Degenerate nucleotide sequences are polynucleotides that encode a polypeptide described herein or fragments thereof, but differ in nucleic acid sequence from the wild-type polynucleotide sequence, due to the degeneracy of the genetic code. Complementary DNA (cDNA) molecules, species homologs, and variants of polynucleotides that encode biologically functional polypeptides also are polynucleotides.

Polynucleotides can comprise coding sequences for naturally occurring polypeptides or can encode altered sequences that do not occur in nature.

Unless otherwise indicated, the term polynucleotide or gene includes reference to the specified sequence as well as the complementary sequence thereof.

The expression products of genes or polynucleotides are often proteins, or polypeptides, but in non-protein coding genes such as rRNA genes or tRNA genes, the product is a functional RNA. The process of gene expression is used by all known life forms, i.e., eukaryotes (including multicellular organisms), prokaryotes (bacteria and archaea), and viruses, to generate the macromolecular machinery for life. Several steps in the gene expression process can be modulated, including the transcription, up-regulation, RNA splicing, translation, and post-translational modification of a protein.

Polypeptides

A polypeptide is a polymer of two or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide, etc., has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure. A purified polypeptide does not include unpurified or semi-purified cell extracts or mixtures of polypeptides that are less than 70% pure.

The term “polypeptides” can refer to one or more of one type of polypeptide (a set of polypeptides). “Polypeptides” can also refer to mixtures of two or more different types of polypeptides (a mixture of polypeptides). The terms “polypeptides” or “polypeptide” can each also mean “one or more polypeptides.”

As used herein, the term “polypeptide of interest” or “polypeptides of interest”, “protein of interest”, “proteins of interest” includes any or a plurality of any of the TDP-43 polypeptides or other polypeptides (including fragment polypeptides) described herein. For example, a polypeptide of interest can be a TDP-43 polypeptide fragment.

A mutated protein or polypeptide comprises at least one deleted, inserted, and/or substituted amino acid, which can be accomplished via mutagenesis of polynucleotides encoding these amino acids. Mutagenesis includes well-known methods in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989).

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Variants will be sufficiently similar to the amino acid sequence of the polypeptides described herein. Such variants generally retain the functional activity of the polypeptides described herein. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

Polypeptides and polynucleotides that are sufficiently similar to polypeptides and polynucleotides described herein (e.g., TDP-43 polypeptide or polypeptide fragment thereof) can be used herein. Polypeptides and polynucleotides that are about 85, 90, 91, 92, 93, 94 95, 96, 97, 98, 99 99.5% or more homologous or identical to polypeptides and polynucleotides described herein (e.g., TDP-43 polypeptide fragment, and variants thereof) can also be used herein.

Expression Cassettes

A recombinant construct is a polynucleotide having heterologous polynucleotide elements. Recombinant constructs include expression cassettes or expression constructs, which refer to an assembly that is capable of directing the expression of a polynucleotide or gene of interest. An expression cassette generally includes regulatory elements such as a promoter that is operably linked to (so as to direct transcription of) a polynucleotide and often includes a polyadenylation sequence as well.

An expression cassette can comprise a fragment of DNA comprising a coding sequence of a selected polypeptide (e.g. TDP-43 polypeptide fragment, or combinations thereof) and regulatory elements preceding (5′ non-coding sequences) and following (3′ non-coding sequences) the coding sequence that are required for expression of the selected gene product. Thus, an expression cassette is typically composed of: 1) a promoter sequence; 2) one or more coding sequences [“ORF”]; and, 3) a 3′ untranslated region (i.e., a terminator) that, in eukaryotes, usually contains a polyadenylation site. Expression cassettes can be circular or linear nucleic acid molecules.

A recombinant construct or expression cassette can be contained within a vector, to facilitate cloning and transformation. In addition to the components of the recombinant construct, the vector can include, one or more selectable markers, a signal which allows the vector to exist as single-stranded DNA (e.g., a M13 origin of replication), at least one multiple cloning site, and a origin of replication (e.g., a SV40 or adenovirus origin of replication). Different expression cassettes can be transformed into different organisms including bacteria, yeast, plants, and mammalian cells, as long as the correct regulatory elements are used for each host.

Generally, a polynucleotide or gene that is introduced into a genetically engineered organism is part of a recombinant construct. A polynucleotide can comprise a gene of interest, e.g., a coding sequence for a protein, or can be a sequence that is capable of regulating expression of a gene, such as a regulatory element, an antisense sequence, a sense suppression sequence, or a miRNA sequence. A recombinant construct can include, for example, regulatory elements operably linked 5′ or 3′ to a polynucleotide encoding one or more polypeptides of interest. For example, a promoter can be operably linked with a polynucleotide encoding one or more polypeptides of interest when it is capable of affecting the expression of the polynucleotide (i.e., the polynucleotide is under the transcriptional control of the promoter). Polynucleotides can be operably linked to regulatory elements in sense or antisense orientation. The expression cassettes or recombinant constructs can additionally contain a 5′ leader polynucleotide. A leader polynucleotide can contain a promoter as well as an upstream region of a gene. The regulatory elements (i.e., promoters, enhancers, transcriptional regulatory regions, translational regulatory regions, and translational termination regions) and/or the polynucleotide encoding a signal anchor can be native/analogous to the host cell or to each other. Alternatively, the regulatory elements can be heterologous to the host cell or to each other. See, U.S. Pat. No. 7,205,453 and U.S. Patent Application Publication Nos. 2006/0218670 and 2006/0248616. The expression cassette or recombinant construct can additionally contain one or more selectable marker genes.

Methods for preparing polynucleotides operably linked to a regulatory elements and expressing polypeptides in a host cell are well-known in the art. See, e.g., U.S. Pat. No. 4,366,246. A polynucleotide can be operably linked when it is positioned adjacent to or close to one or more regulatory elements, which direct transcription and/or translation of the polynucleotide.

Promoter

A promoter is a nucleotide sequence that is capable of controlling the expression of a coding sequence or gene. Promoters are generally located 5′ of the sequence that they regulate. Promoters can be derived in their entirety from a native gene, or be composed of different elements derived from promoters found in nature, and/or comprise synthetic nucleotide segments. Those skilled in the art will readily ascertain that different promoters can regulate expression of a coding sequence or gene in response to a particular stimulus, e.g., in a cell- or tissue-specific manner, in response to different environmental or physiological conditions, or in response to specific compounds. Promoters are typically classified into two classes: inducible and constitutive. A constitutive promoter refers to a promoter that allows for continual transcription of the coding sequence or gene under its control.

An inducible promoter refers to a promoter that initiates increased levels of transcription of the coding sequence or gene under its control in response to a stimulus or an exogenous environmental condition. If inducible, there are inducer polynucleotides present therein that mediate regulation of expression so that the associated polynucleotide is transcribed only when an inducer molecule is present. A directly inducible promoter refers to a regulatory region, wherein the regulatory region is operably linked to a gene encoding a protein or polypeptide, where, in the presence of an inducer of the regulatory region, the protein or polypeptide is expressed. An indirectly inducible promoter refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by inducible promoter.

A promoter can be any polynucleotide that shows transcriptional activity in the chosen host microorganism. A promoter can be naturally-occurring, can be composed of portions of various naturally-occurring promoters, or may be partially or totally synthetic. Guidance for the design of promoters is derived from studies of promoter structure, such as that of Harley and Reynolds, Nucleic Acids Res., 15, 2343-61 (1987). In addition, the location of the promoter relative to the transcription start can be optimized. Many suitable promoters for use in mammalian cells are well known in the art, as are polynucleotides that enhance expression of an associated expressible polynucleotide. Non-limiting examples of constitutive promoters that can be used to in the present expression cassette can include cytomegalovirus (CMV) promoter and the Rous sarcoma virus promoter, chicken beta actin promoter, ubiquitin promoter, that allows for unregulated expression in mammalian cells.

Fluorescent Proteins

Fluorescent proteins are proteins characterized by their ability to absorb light at a certain wavelength (excitation), and to subsequently emit of secondary fluorescence at a longer wavelength (emission), which can be detected. The excitation and emission wavelengths are often separated from each other by tens to hundreds of nanometers.

In an embodiment, the fluorescent proteins can be the members of a FRET pair.

Fluorescence resonance energy transfer or FRET, can be used to determine if two fluorescent proteins are within a certain distance of each other. By using a mechanism relying on energy transfer between two light-sensitive fluorescent proteins, the interaction, or lack thereof, between two molecules can be detected; therefore allowing the extremely sensitive detection of small changes in the distance between two molecules. The fundamental mechanism of FRET involves a donor fluorescent protein in an excited electronic state, which can transfer its excitation energy to a nearby acceptor fluorescent protein through a non-radiative long-range dipole-dipole interactions. The efficiency of this energy transfer being inversely proportional to the sixth power of the distance between donor and acceptor fluorescent proteins.

In the presence of a suitable acceptor, the donor fluorescent protein can transfer excited state energy directly to the acceptor without emitting a photon. The resulting fluorescence sensitized emission has characteristics similar to the emission spectrum of the acceptor.

A FRET proximity detection protein pair can comprise a donor fluorescent protein and a acceptor fluorescent protein having compatible excitation and emission wavelength, to allow the detection of an energy transfer.

Non-limiting examples of FRET proximity detection protein pairs include mClover3/mRuby3, EBFP2/mEGFP, ECFP/EYFP, CeruleanNenus, MiCy/mKO, CyPet/YPet, EGFP/mCherry, Venus/mCherry, Venus/tdTomato, and Venus/m Plum.

In another embodiment, any proximity detection system for proteins, including fluorescence complementation, bioluminescence resonance energy transfer, split luciferase assay and Split-APEX2 can be used.

Polynucleotides encoding fluorescent proteins can be incorporated into expression cassette. Polynucleotides encoding fluorescent proteins can be operably linked to a promoter, for their own expression, or operably linked to a polynucleotide encoding a protein of interest, for the expression of a fusion protein comprising the protein of interest and the fluorescent protein. In an embodiment, a TDP-43 polypeptide fragment can be operably linked to a fluorescent protein that is a member of a proximity detection pair.

Linkers

Methods for attaching two individual elements can require the use of a linker to create a bond between two molecules thought to be conjugated or fused to one another. Fusion proteins result from the fusion two or more protein domains together, and each protein or protein domain can be fused to the next using a linker. Suitable linkers for the fusion of two or more protein or protein domains can include natural linkers, and empirical linkers.

Natural linkers can be derived from multi-domain proteins, which are naturally present between protein domains. Natural linkers can have several properties depending or their such as length, hydrophobicity, amino acid residues, and secondary structure, which can impact the fusion protein in different way.

The studies of linkers in natural multi-domain proteins have led to the generation of many empirical linkers with various sequences and conformations for the construction of recombinant fusion proteins. Empirical linkers can be classified in three types: flexible linkers, rigid linkers, and cleavable linkers. Flexible linkers can provide a certain degree of movement or interaction at the joined domains. They are generally composed of small, non-polar (e.g. Gly) or polar (e.g. Ser or Thr) amino acids, which provides flexibility, and allows for mobility of the connecting functional domains. Rigid linkers can successfully keep a fixed distance between the domains to maintain their independent functions, which can provide efficient separation of the protein domains or sufficient reduction of their interference with each other. Cleavable linkers can allow the release of functional domains in vivo. By taking advantage of unique in vivo processes, they can be cleaved under specific conditions such as the presence of reducing reagents or proteases. This type of linker can reduce steric hindrance, improve bioactivity, or achieve independent actions/metabolism of individual domains of recombinant fusion proteins after linker cleavage. Non-limiting examples of empiric linkers can include those listed in Table 1.

TABLE 1
Eamples of empiric linkers

| | Linker | | |
|---|---|---|---|
| Linker Function | Type | Sequence | SEQ ID NO: |
| Increase | flexible | (GGGGS)₃ | SEQ ID NO: 9 |
| Stability/ | flexible | (Gly)₈ | SEQ ID NO: 10 |
| Folding | | | |
| | flexible | (Gly)₆ | SEQ ID NO: 11 |
| | rigid | (EAAAK)₃ | SEQ ID NO: 12 |
| | rigid | (EAAAK)ₙ (n = 1-3) | SEQ ID NO: 12 |
| Increase | rigid | A(EAAAK)₄ALEA | SEQ ID NO: 13 |
| expression | | (EAAAK)₄A | |
| Improve | flexible | (GGGGS)₃ | SEQ ID NO: 9 |
| biological | | | |
| activity | | | |
| | rigid | A(EAAAK)₄ALEA(EAAAK)₄A | SEQ ID NO: 13 |
| | flexible | GGGGS | SEQ ID NO: 14 |
| | rigid | PAPAP | SEQ ID NO: 15 |
| | rigid | AEAAAKEAAAKA | SEQ ID NO: 16 |
| | flexible | (GGGGS)ₙ (n = 1, 2, 4) | SEQ ID NO: 17 |
| | rigid | (Ala-Pro)ₙ (10-34 aa) | SEQ ID NO: 18 |
| | cleavable | disulfide | N/A |
| | cleavable | disulfide | N/A |
| Enable | cleavable | VSQTSKLTR↓AETVFPDVD^{b} | SEQ ID NO: 19 |
| targeting | | | |
| | cleavable | PLG ↓ LWA^{c} | SEQ ID NO: 20 |
| | cleavable | RVL↓AEA; EDVVCC↓SMSY; | SEQ ID NO: 21 |
| | | GGIEGR↓GS^{c} | SEQ ID NO: 22 |
| | | | SEQ ID NO: 23 |
| | cleavable | TRHRQPR↓GWE; | SEQ ID NO: 24 |
| | | AGNRVRR↓SVG; | SEQ ID NO: 25 |
| | | RRRRRRR↓R↓R^{d} | SEQ ID NO: 26 |
| | cleavable | GFLG↓^{e} | SEQ ID NO: 27 |
| Alter PK | dipeptide | LE | N/A |
| | rigid | A(EAAAK)₄ALEA(EAAAK)₄A | SEQ ID NO: 13 |
| | cleavable | Disulfide | N/A |

“↓” Protease sensitive cleavage sites;
^{b}Factor Xla/FVIla sensitive cleavage;
^{c}Matrix metalloprotease-1 sensitive cleavage sequences;
^{d}HIV PR (HIV-1 protease); NS3 protease (HCV protease); Factor Xa sensitive cleavage, respectively;
^{e}Furin sensitive cleavage;
^{f}Cathepsin B sensitive cleavage

The procedures described herein employ, unless otherwise indicated, conventional techniques of chemistry, molecular biology, microbiology, recombinant DNA, genetics, immunology, cell biology, cell culture and transgenic biology, which are within the skill of the art. (See, e.g., Maniatis, et al., Molecular Cloning, Cold Spring Harbor Laboratory Press, Cold Spring Harbor, N.Y. (1982); Sambrook et al., (1989); Sambrook and Russell, Molecular Cloning, 3rd Ed., Cold Spring Harbor Laboratory Press, Cold Spring Harbor, N.Y. (2001); Ausubel, et al., Current Protocols in Molecular Biology, John Wiley & Sons (including periodic updates) (1992); Glover, DNA Cloning, IRL Press, Oxford (1985); Russell, Molecular biology of plants: a laboratory course manual, Cold Spring Harbor Laboratory Press, Cold Spring Harbor, N.Y. (1984); Anand, Techniques for the Analysis of Complex Genomes, Academic Press, N Y (1992); Guthrie and Fink, Guide to Yeast Genetics and Molecular Biology, Academic Press, N Y (1991); Harlow and Lane, Antibodies, Cold Spring Harbor Laboratory Press, Cold Spring Harbor, N.Y. (1988); Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, A. R. Liss, Inc. (1987); Immobilized Cells And Enzymes, IRL Press (1986); B. Perbal, A Practical Guide To Molecular Cloning (1984); the treatise, Methods In Enzymology, Academic Press, Inc., NY); Methods In Enzymology, Vols. 154 and 155, Wu, et al., eds.; Immunochemical Methods In Cell And Molecular Biology, Mayer and Walker, eds., Academic Press, London (1987); Handbook Of Experimental Immunology, Volumes I-IV, D. M. Weir and C. C. Blackwell, eds. (1986); Riott, Essential Immunology, 6th Edition, Blackwell Scientific Publications, Oxford (1988); Fire, et al., RNA Interference Technology From Basic Science to Drug Development, Cambridge University Press, Cambridge (2005); Schepers, RNA Interference in Practice, Wiley-VCH (2005); Engelke, RNA Interference (RNA): The Nuts & Bolts of siRNA Technology, DNA Press (2003); Gott, RNA Interference, Editing, and Modification: Methods and Protocols (Methods in Molecular Biology), Human Press, Totowa, N.J. (2004); and Sohail, Gene Silencing by RNA Interference: Technology and Application, CRC (2004)).

In an embodiment an expression cassette can comprise one or more polynucleotides encoding a TDP-43 polypeptide fragment. One or more TDP-43 polypeptide fragments can be identical TDP-43 polypeptide fragments, or non-identical TDP-43 polypeptide fragments having different lengths, but that can co-assemble.

A TDP-43 protein comprises an unstructured C-terminal domain encompassing residues from about 274 to about 414 (CTD), which comprises a glycine-rich region, involved in protein-protein interactions. Two or more TDP-43 fragments as described herein can each comprise a CTD domain. These fragments can interact with one another (i.e., co-assemble or aggregate with one another). That is the fragments can have close contact with one another. As used herein, a “TDP-43 polypeptide fragment” refers to a fragment of a TDP-43 polypeptide, that retains at least a portion of a CTD domain, and which allows it to interact and co-assemble or aggregate with another TDP-43 polypeptide fragment. “TDP-43 polypeptide fragment” therefore refers to any TDP-43 polypeptide fragment that can interact and co-assemble with another TDP-43 polypeptide fragment regardless of the length of the fragment, as long as both TDP-43 polypeptide fragments comprise one or more CTD domains. Therefore, a TDP-43 polypeptide fragment can interact with and co-assemble or aggregate with an identical TDP-43 polypeptide fragment (e.g., having a same length and a CTD domain), or with a non-identical TDP-43 polypeptide fragment (e.g., having a different length, but comprising a CTD domain).

For example, a TDP-43 polypeptide fragment can comprise a sequence as set forth in SEQ ID NO:4 (amino acids 262-414 of a TDP-43 protein) or as set forth in SEQ ID NO:8 (amino acids 274-414 of a TDP-43 protein). Other examples include fragments comprising from about amino acid 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, or 285 to about amino acid 395, 396, 397, 398, 399, 400, 401, 402, 403, 404, 405, 406, 407, 408, 409, 410, 411, 412, 413, or 414. A TDP-43 polypeptide fragment can be about 110, 115, 120, 125, 130, 135, 140, 145, 150, 152, 155, 160, or 164 amino acids in length. The sequence of full-length TDP-43 is shown below (SEQ ID NO:28). A TDP-43 polypeptide fragment, e.g. a fragment comprising a sequence as set forth in SEQ ID NO:4 can interact and co-assemble with another identical TDP-43 polypeptide fragment (e.g., SEQ ID NO:4) or with a different TDP-43 polypeptide fragment (e.g., SEQ ID NO:8). A TDP-43 polypeptide fragment, e.g. a fragment comprising a sequence as set forth in SEQ ID NO:8 can interact and co-assemble with another identical TDP-43 polypeptide fragment (e.g., SEQ ID NO:8) or with a different a TDP-43 polypeptide fragment (e.g., SEQ ID NO:4).

In an embodiment, an expression cassette can comprise TDP-43 polynucleotides as set forth in SEQ ID NO:3 or SEQ ID NO:7. In another embodiment, the polynucleotides can encode TDP-43 polypeptides comprising sequences as set forth in SEQ ID NO:4 or 8.

An expression cassette can further comprise one or more polynucleotides encoding a promoter and one or more polynucleotides encoding a fluorescent protein. An expression cassette can further comprise one or more polynucleotides encoding a linker sequence. The linker sequence can be present between a polynucleotide encoding a fluorescent protein and a polynucleotide encoding a polypeptide comprising a sequence as set forth in SEQ NO:4 or SEQ ID NO:8. A fluorescent protein can be a fluorescent donor protein or a fluorescent acceptor protein of a proximity detection protein pair.

In an embodiment, an expression cassette can comprise a polynucleotide encoding a promoter, a polynucleotide encoding a TDP-43 polypeptide fragment comprising a sequence as set forth in SEQ ID NO:3, a polynucleotide encoding a linker sequence, and a polynucleotide encoding a donor fluorescent protein.

In another embodiment, an expression cassette can comprise a polynucleotide encoding a promoter, a polynucleotide encoding a TDP-43 polypeptide fragment comprising a sequence as set forth in SEQ ID NO:3, a polynucleotide encoding a linker sequence, and a polynucleotide encoding an acceptor fluorescent protein.

Polynucleotides can be operably linked to one another. For example a polynucleotide encoding a promoter can be operably linked to a polynucleotide encoding a TDP-43 polypeptide fragment. A polynucleotide encoding a promoter can be operably linked to a polynucleotide encoding a donor or acceptor fluorescent protein. A polynucleotide encoding a TDP-43 polypeptide fragment can be operably linked to a polynucleotide encoding a donor or acceptor fluorescent protein. A linker can be operably linked to a polynucleotide encoding a donor or acceptor fluorescent protein and/or to a polynucleotide encoding a TDP-43 polypeptide fragment.

In another embodiment, an expression cassette can comprise a first polynucleotide encoding a first promoter, a polynucleotide encoding a TDP-43 polypeptide fragment (e.g. SEQ ID NO:3), a polynucleotide encoding a linker sequence, and a polynucleotide encoding a donor fluorescent protein; and a second polynucleotide encoding a second promoter, a polynucleotide encoding a TDP-43 polypeptide fragment (e.g. SEQ ID NO:3), a polynucleotide encoding a linker sequence, and a polynucleotide encoding a acceptor fluorescent protein.

In an embodiment, a first polynucleotide encoding a first promoter can be operably linked to a polynucleotide encoding a TDP-43 polypeptide fragment (e.g. SEQ ID NO:3), operably linked to a polynucleotide encoding a linker sequence, operably linked to a polynucleotide encoding a donor fluorescent protein. The first polynucleotide can be operably linked to a second polynucleotide. The second polynucleotide can encode a second promoter operably linked to a TDP-43 polypeptide fragment (e.g. SEQ ID NO:3), operably linked to a polynucleotide encoding a linker sequence, which can be operably linked to a polynucleotide encoding an acceptor fluorescent protein.

A first and a second promoter can be a same promoter, or a first and a second promoter can be two different promoters.

In an embodiment, an expression cassette can comprise a polynucleotide encoding a promoter, a polynucleotide encoding a TDP-43 polypeptide fragment comprising a sequence as set forth in SEQ ID NO:7, a polynucleotide encoding a linker sequence, a polynucleotide encoding a donor fluorescent protein.

In another embodiment, an expression cassette can comprise a polynucleotide encoding a promoter, a polynucleotide encoding a TDP-43 polypeptide fragment comprising a sequence as set forth in SEQ ID NO:7, a polynucleotide encoding a linker sequence, and a polynucleotide encoding an acceptor fluorescent protein.

In an embodiment An expression cassette can comprise a first polynucleotide encoding a first promoter, a polynucleotide encoding a TDP-43 polypeptide fragment (e.g. SEQ ID NO:7), a polynucleotide encoding a linker sequence, and a polynucleotide encoding a donor fluorescent protein; and a second polynucleotide encoding a second promoter, a polynucleotide encoding a TDP-43 polypeptide fragment (e.g. SEQ ID NO:7), a polynucleotide encoding a linker sequence, and a polynucleotide encoding an acceptor fluorescent protein.

In an embodiment, a first polynucleotide encoding a first promoter can be operably linked to a polynucleotide encoding a TDP-43 polypeptide fragment (e.g. SEQ ID NO:7), operably linked to a polynucleotide encoding a linker sequence, operably linked to a polynucleotide encoding a donor fluorescent protein. The first polynucleotide can be operably linked to a second polynucleotide. The second polynucleotide can encode a second promoter operably linked to a TDP-43 polypeptide fragment (e.g. SEQ ID NO:7), operably linked to a polynucleotide encoding a linker sequence, which can be operably linked to a polynucleotide encoding an acceptor fluorescent protein.

Vectors

An expression cassette can be delivered to cells (e.g., a plurality of different cells or cell types including target cells or cell types and/or non-target cell types) in a vector (e.g., an expression vector). A vector can be an integrating or non-integrating vector, referring to the ability of the vector to integrate the expression cassette and/or transgene into a genome of a cell. Either an integrating vector or a non-integrating vector can be used to deliver an expression cassette containing one or more polypeptides described herein. Examples of vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs); episomal vectors; transposons (e.g., PiggyBac); and (b) viral vectors such as retroviral vectors, lentiviral vectors, adenoviral vectors, and AAV vectors. Viruses have several advantages for delivery of nucleic acids, including high infectivity and/or tropism for certain target cells or tissues. In some cases, a virus is used to deliver a nucleic acid molecule or expression cassette comprising one or more regulatory elements, as described herein, operably linked to a gene.

In an embodiment, the vector is a lentiviral vector. Lentiviral vectors rely on Lentivirus for the infection and incorporation of genetic material into a host cell. Lentivirus is a genus of retroviruses characterized by long incubation periods. The best known lentivirus is the human immunodeficiency virus (HIV), which causes AIDS. Lentiviruses can integrate a significant amount of DNA into the DNA of the host cell and can efficiently infect nondividing cells, so they are one of the most efficient methods of gene delivery. Lentiviruses can become endogenous, integrating their genome into the host germline genome, so that the virus is henceforth inherited by the host's daughter cells during cellular division. Lentiviral infection has advantages over other viral and non-viral vectors, including high-efficiency infection of dividing and non-dividing cells, long-term stable expression of a transgene, and low immunogenicity. Non-limiting examples of lentiviral vectors include vector derived from lentiviruses such as human immunodeficiency virus (HIV), simian immunodeficiency virus (SIV) and feline immunodeficiency virus (FIV),

Vectors for stable transformation of mammalian are well known in the art and can be obtained from commercial vendors or constructed from publicly available sequence information. Expression vectors can be engineered to produce protein(s) of interest (e.g., TDP-43 fragments). Such vectors are useful for recombinantly producing a protein of interest and for modifying the natural phenotype of host cells.

If desired, polynucleotides can be cloned into an expression vector comprising expression control elements, including for example, origins of replication, promoters, enhancers, or other regulatory elements that drive expression of the polynucleotides in host cells. An expression vector can be, for example, a plasmid, such as pBR322, pUC, or ColE1, or an adenovirus vector, such as an adenovirus Type 2 vector or Type 5 vector. Optionally, other vectors can be used, including but not limited to Sindbis virus, simian virus 40, alphavirus vectors, poxvirus vectors, and cytomegalovirus and retroviral vectors, such as murine sarcoma virus, mouse mammary tumor virus, Moloney murine leukemia virus, and Rous sarcoma virus. Mini-chromosomes such as MC and MC1, bacteriophages, phagemids, yeast artificial chromosomes, bacterial artificial chromosomes, virus particles, virus-like particles, cosmids (plasmids into which phage lambda cos sites have been inserted) and replicons (genetic elements that are capable of replication under their own control in a cell) can also be used.

To confirm the presence of recombinant polynucleotides or recombinant genes in transgenic cells, a polymerase chain reaction (PCR) amplification or Southern blot analysis can be performed using methods known to those skilled in the art. Expression products of the recombinant polynucleotides or recombinant genes can be detected in any of a variety of ways, and include for example, western blot and enzyme assay. Once recombinant organisms have been obtained, they may be grown in cell culture.

Techniques contemplated herein for gene expression in mammalian cells can include delivery via a viral vector (e.g., retroviral, adenoviral, AAV, helper-dependent adenoviral systems, hybrid adenoviral systems, herpes simplex, pox virus, lentivirus, and Epstein-Barr virus), and non-viral systems, such as physical systems (naked DNA, DNA bombardment, electroporation, hydrodynamic, ultrasound, and magnetofection), and chemical system (cationic lipids, different cationic polymers, and lipid polymers).

For example, vectors described herein can be introduced into a cell to be altered thus allowing expression of the recombinant protein using any of the variety of methods that are known in the art and suitable for introduction of nucleic acid molecule into a cell. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as Lipofectamine™ Dojindo Hilymax™, Fugene™, jetPEI™, Effectene™ and DreamFect™.

In an embodiment, a vector can comprise a polynucleotide encoding a promoter, a polynucleotide encoding a TDP-43 polypeptide fragment, a polynucleotide encoding a linker sequence, and a polynucleotide encoding a donor fluorescent protein. A polynucleotide encoding a TDP-43 polypeptide fragment can encode, for example, a polypeptide comprising a sequence as set forth in SEQ ID NO:4 or 8. In another example, a vector can comprise a polynucleotide as set forth in SEQ ID NO:1 or SEQ ID NO:5. Polynucleotides within the vector can be operably linked to one another.

In another embodiment, a vector can comprise a polynucleotide encoding a promoter, a polynucleotide encoding a TDP-43 polypeptide fragment, a polynucleotide encoding a linker sequence, and a polynucleotide encoding an acceptor fluorescent protein. A polynucleotide encoding a TDP-43 polypeptide fragment can encode a polypeptide comprising, for example, a sequence as set forth in SEQ ID NO:4 or 8. In another example, a vector can comprise a polynucleotide as set forth in SEQ ID NO:2 or SEQ ID NO:6. Polynucleotides within the vector can be operably linked to one another.

In another embodiment a vector can comprise a first polynucleotide encoding a first promoter, a polynucleotide encoding a TDP-43 polypeptide fragment, a polynucleotide encoding a linker sequence, and a polynucleotide encoding a donor fluorescent protein; a second polynucleotide encoding a second promoter, a polynucleotide encoding a TDP-43 polypeptide fragment, a polynucleotide encoding a linker sequence, and a polynucleotide encoding an acceptor fluorescent protein. Polynucleotides within the vector can be operably linked to one another.

A first polynucleotide encoding a TDP-43 polypeptide fragment can be identical to a TDP-43 polypeptide fragment in the second polynucleotide, or can be a non-identical TDP-43 polypeptide fragment having different length, but that can co-assemble with a TDP-43 polypeptide fragment in the first polynucleotide. For example, a first polynucleotide encoding a TDP-43 polypeptide fragment can comprise SEQ ID NO:4 or 8, and a second TDP-43 polypeptide fragment can comprise SEQ ID NO:4 or 8.

Host Cells

Vectors described herein can be introduced into host cell to be altered thus allowing expression of recombinant, heterologous polypeptides within the cell. A variety of host cells are known in the art and suitable for recombinant proteins expression. Examples of typical cells used for transfection include, but are not limited to, a bacterial cell, a eukaryotic cell, a yeast cell, an insect cell, a mammalian cell or a plant cell. Non-limiting examples of host cells can include, E. coli, Bacillus, Streptomyces, Pichia pastoris, Salmonella typhimurium, Drosophila S2, Spodoptera SJ9. A mammalian cell can include, for example a cell derived from a rodent (such as a mice, a rat, or a hamster), a primate (such as a monkey, or a human). Mammalian cells can be derived from a healthy tissue, or from a diseased tissue such as a tumor. Mammalian cells can be immortalized, to ensure non-limiting cell growth in culture. Non-limiting examples of mammalian host cells can include, CHO, COS (e.g. COS-7), 3T3-F442A, HeLa, HUVEC, HUAEC, NIH 3T3, Jurkat, HEK293, HEK293H, or HEK293F.

In an embodiment, the host cell can be a HeLa cell. HeLa is an immortal cell line widely used in scientific research. It is the oldest and most commonly used human cell line, that was derived from cervical cancer cells in 1951. The cell line was found to be remarkably durable and prolific, as compared to cells cultured from other human cells, which would only survive for a few days.

A host cell can comprise one or more expression cassettes. A host cell can comprise one or more vectors comprising one or more heterologous polynucleotides not present in a corresponding wild-type cell. In an embodiment, a host cell does not naturally comprise the vectors or expression cassettes.

An embodiment provides a host cell comprising an expression cassette comprising one or more polynucleotides encoding a TDP-43 polypeptide fragment (e.g., SEQ ID NO:4 or 8).

Another embodiment provides a host cell comprising a first vector comprising a polynucleotide encoding a first TDP-43 polypeptide fragment and a polynucleotide encoding a fluorescent donor protein, and a second vector comprising a polynucleotide encoding a second TDP-43 polypeptide fragment and a polynucleotide encoding a fluorescent acceptor protein. A first TDP-43 polypeptide fragment and a second TDP-43 polypeptide fragment can be identical TDP-43 polypeptide fragments, or non-identical TDP-43 polypeptide fragments having different lengths, but that can co-assemble with one another (e.g., both can comprise a CTD domain, or a fragment thereof, which contains a glycine-rich region involved in protein-protein interactions of TDP-43 peptide).

For example, a host cell can comprise a first vector comprising a polynucleotide encoding a TDP-43 polypeptide fragment (e.g., SEQ ID NO:4), and a donor fluorescent protein (e.g. mRuby3), and a second vector comprising a polynucleotide encoding a TDP-43 polypeptide fragment (e.g., SEQ ID NO:4), and an acceptor fluorescent protein (e.g., a mClover3). In one embodiment, a polynucleotide encoding a TDP-43 polypeptide fragment (e.g. SEQ ID NO:4), can be operably linked to a donor fluorescent protein (e.g. mRuby3), and a polynucleotide encoding a TDP-43 polypeptide fragment (e.g. SEQ ID NO:4), can be operably linked to an acceptor fluorescent protein (e.g. mClover3). A host cell can comprise, for example, a first vector comprising a polynucleotide comprising SEQ ID NO:1 and a second vector comprising a polynucleotide as set forth in SEQ ID NO:2.

A host cell can comprise a first vector comprising a polynucleotide encoding a TDP-43 polypeptide fragment (e.g. SEQ ID NO:8) and a donor fluorescent protein (e.g. mRuby3), and a second vector comprising a polynucleotide encoding a TDP-43 polypeptide fragment (e.g. SEQ ID NO:8) and an acceptor fluorescent protein (e.g. mClover3). In an embodiment, a polynucleotide encoding a TDP-43 polypeptide fragment (e.g. SEQ ID NO:8), can be operably linked to a donor fluorescent protein (e.g. mRuby3), and a polynucleotide encoding a TDP-43 polypeptide fragment (e.g. SEQ ID NO:8), can be operably linked to an acceptor fluorescent protein (e.g. mClover3). A host cell can comprise, for example, a first vector comprising a polynucleotide as set forth in SEQ ID NO:5, and a second vector comprising a polynucleotide as set forth in SEQ ID NO:6.

A host cell can comprise a first vector comprising a polynucleotide encoding a TDP-43 polypeptide fragment (e.g. SEQ ID NO:4) and a donor fluorescent protein (e.g. mRuby3), and a second vector comprising a polynucleotide encoding a TDP-43 polypeptide fragment (e.g. SEQ ID NO:8) and an acceptor fluorescent protein (e.g. mClover3). In an embodiment, a polynucleotide encoding a TDP-43 polypeptide fragment (e.g. SEQ ID NO:4), can be operably linked to a donor fluorescent protein (e.g. mRuby3), and a polynucleotide encoding a TDP-43 polypeptide fragment (e.g. SEQ ID NO:8), can be operably linked to an acceptor fluorescent protein (e.g. mClover3). A host cell can comprise, for example, a first vector comprising a polynucleotide as set forth in SEQ ID NO:1 and a second vector comprising a polynucleotide as set forth in SEQ ID NO:6.

A host cell can comprise a first vector comprising a polynucleotide encoding a TDP-43 polypeptide fragment (e.g. SEQ ID NO:8) and a donor fluorescent protein (e.g., a mRuby3 donor fluorescent protein) and a second vector comprising a polynucleotide encoding a TDP-43 polypeptide fragment (e.g. SEQ ID NO:4) and an acceptor fluorescent protein (e.g., a mClover3 acceptor fluorescent protein). In an embodiment, a polynucleotide encoding a TDP-43 polypeptide fragment (e.g. SEQ ID NO:8), can be operably linked to a donor fluorescent protein (e.g. mRuby3), and a polynucleotide encoding a TDP-43 polypeptide fragment (e.g. SEQ ID NO:4), can be operably linked to an acceptor fluorescent protein (e.g. mClover3). A host cell can comprise, for example, a first vector comprising a polynucleotide as set forth in SEQ ID NO:5 and a second vector comprising a polynucleotide as set forth in SEQ ID NO:2.

Methods of Identifying TDP-43 Peptides in a Sample

An embodiment provides methods of measuring a titer of TDP-43 polypeptides in a sample or detecting a TDP-43 polypeptides or aggregates in a sample.

A sample can be contacted with a host cell comprising with one or more vectors described herein. The host cells can be exposed to an excitation light. An emission light signal can be detected.

A host cell can be cultured under any suitable culture conditions, and contacted with a test sample. A cell can be exposed to a laser or other suitable light source producing a excitation light corresponding to the excitation wavelength of the donor fluorescent protein. For example, a laser or other suitable light source producing an excitation light between 485-588 nm can be used. Light can then be collected at a wavelength corresponding to the emission wavelength of the acceptor fluorescent protein, which corresponds to the emission light signal. For example, light can be collected between 500-670 nm for the detection of emission light signal.

For mClover3, a 488 nm laser excitation can used, with 500-600 nm light collection. For mRuby3, a 559 nm laser excitation can be used, with 570-670 nm light collected.

Depending on the technology used to collect emission light signals (fluorescent microscopy, flow cytometry, or other suitable method for example), several images can be taken of the cells, and/or multiple cells can be analyzed.

A donor fluorescent protein, in an excited state energy can transfer energy directly to an acceptor fluorescent protein in close proximity without emitting a photon. The resulting fluorescence sensitized emission has characteristics similar to the emission spectrum of the acceptor, and can be detected by immunofluorescent microscopy or by flow cytometry, for example. Any other suitable means for the detection of fluorescence can also be used.

A host cell comprising one or more vectors described herein can express a TDP-43 polypeptide fragment linked or fused to a donor fluorescent protein and a TDP-43 polypeptide fragment linked or fused to an acceptor fluorescent protein. The fragments can aggregate with one another to form TDP-43 aggregates, which can bring a donor fluorescent protein in close proximity to an acceptor fluorescent protein. Upon exposition of such host cell to an excitation light, energy from a donor fluorescent protein can be transferred to an acceptor fluorescent protein, which can emit emission light signal that can be detected

In the presence of exogenous TDP-43 polypeptide fragments or aggregates in a sample (i.e., TDP-43 polypeptide fragments or aggregates not expressed or generated by a host cell), for example providing from a sample comprising TDP-43 polypeptide fragments or aggregates, TDP-43 polypeptide fragments linked to a fluorescent protein, expressed by a host cell, can interact with and form aggregates with exogenous TDP-43 polypeptide fragments or aggregates. Exogenous TDP-43 polypeptide fragments or aggregates can compete with TDP-43 polypeptide fragments linked to fluorescent proteins (either donor or acceptor), and generate aggregates comprising exogenous TDP-43 polypeptide fragment or aggregate and TDP-43 polypeptide fragment linked to a fluorescent protein. A binding competition can result in the generation of a distance between a donor fluorescent protein and an acceptor fluorescent protein, which can prevent an energy transfer from a donor fluorescent protein to an acceptor fluorescent protein, resulting in a reduction or in a lack of emission of a light signal. Therefore, detecting an emission light signal can indicate that a sample does not comprise TDP-43 peptide or aggregate, while a lack of an emission light signal can indicate that a sample comprises TDP-43 peptides or aggregates.

An emission light signal measured in the absence of a sample, or in the presence of a sample known for not containing any TDP-43 polypeptide fragments can be used as a positive control. In such case, nothing disturbs the interaction between a TDP-43 peptide linked to a donor fluorescent protein and a TDP-43 peptide linked to an acceptor fluorescent protein; and a emission light signal can be detected. An emission light signal measured in a cell that does not express a TDP-43 polypeptide fragment linked to a donor fluorescent protein nor a TDP-43 polypeptide fragment linked to an acceptor fluorescent protein (i.e., a cell expressing a TDP-43 polypeptide fragment linked to a donor fluorescent protein only, a cell expressing a TDP-43 polypeptide fragment linked to an acceptor fluorescent protein only, or a cell not expressing any TDP-43 polypeptide fragment) can be used as an internal control, to evaluate any auto-fluorescent signal that can be emitted by a cell. In such case, no emission light signal can be detected as a result of a transfer of energy from a donor fluorescent protein to an acceptor fluorescent protein; and nothing but cell autofluorescence can be detected. Furthermore, an emission light signal measured in the presence of a sample comprising exogenous TDP-43 polypeptide fragment or aggregates can be used as a negative control. In such case, exogenous TDP-43 polypeptide fragment or aggregate can disturb the interaction between a TDP-43 peptide linked to a donor fluorescent protein and a TDP-43 peptide linked to an acceptor fluorescent protein; and a weaker or absent emission light signal can be detected.

If an emission light signal measured in a sample is equivalent to or greater than a positive control, it can indicate that a sample does not comprise TDP-43 polypeptide fragment or aggregate.

If an emission light signal measured in a sample is less than a positive control, or greater than a negative control, it can indicate that a sample does comprise TDP-43 polypeptide fragments or aggregates. Alternatively, if an emission light measured in a sample is greater than or equivalent to a negative control, it can indicate that a sample does comprise TDP-43 polypeptide fragments or aggregates. Similarly, if an emission light measured in a sample is less than or equivalent to a negative control and greater than an internal control, it can indicate that a sample does comprise TDP-43 polypeptide fragments or aggregates.

If an emission light signal measured in a sample is less than an internal control, it can indicate that a test is inconclusive, and no conclusion can be reached regarding the presence or absence of TDP-43 polypeptide fragments or aggregates in a sample.

Binding competition can be weak, if an amount of exogenous TDP-43 polypeptide fragment or aggregate from a sample is small, and can be strong if an amount of exogenous TDP-43 polypeptide fragment or aggregate from a sample is large. An amount of exogenous TDP-43 polypeptide fragment or aggregates can thus modify an amount of emission light signal detected in a dose dependent manner, which can be used to measure a titer to TDP-43 polypeptide fragment or aggregate in a sample. The emission light signal can be compared to a standard curve comparing emission light signals obtained in the presence of predetermined amounts of TDP-43 polypeptide peptide. For example, emission light signals measured in the presence of various concentrations of TDP-43 polypeptide fragment can be used to generate a standard curve.

If an emission light signal measured in a sample is equivalent to or greater than a positive control, it can indicate that the titer of TDP-43 polypeptide fragments or aggregates in the sample is zero.

If an emission light signal measured in a sample is less than a positive control, but greater than a negative control, it can then be compared to the emission light signal in an standard curve, to estimate the concentration (i.e., the titer) of TDP-43 polypeptide fragments or aggregates present in the sample.

Methods of Detecting TDP-43 Related Neuropathological Disease or Condition in a Subject

An embodiment provides methods for detecting TDP-43 related neuropathological disease or condition in a subject.

TDP-43 fragments can aggregate with one another, accumulate, and spread using prion mechanisms of action. TDP-43 polypeptide fragments or aggregates (or TDP-43 prions) are pathological, and can be detected in subjects diagnosed with neurodegenerative diseases, associated with the accumulation of pathological protein in neurons, responsible for neurodegeneration. Therefore, detecting TDP-43 polypeptide fragments or aggregates in a sample collected from a subject, as detailed above, can be used to detect or diagnose a neurological disease or condition associated with the accumulation of TDP-43 polypeptide fragments or aggregates, such as frontotemporal dementia (FTLD), amyotrophic lateral sclerosis (ALS), chronic traumatic encephalopathy, Alzheimer's disease (AD), limbic-predominant age-related (LATE), and TDP-43 encephalopathy.

A “sample” or “test sample” can be collected from a subject, in which the presence of, or the titer of TDP-43 polypeptide fragments or aggregates is sought to be measured. A “test sample” is a sample for which the presence (or absence) of or the titer of TDP-43 fragment polypeptide is sought to be analyzed. The sample can be a biological fluid, a tissue sample, or an aggregated material amplified in vitro therefrom.

A sample can be prepared in any suitable way to can facilitate, enhance, or improve the detection or measurement of TDP-43 polypeptide fragments or aggregates. For example, a sample can be concentrated, or diluted; material present in the sample can be amplified (using a protein-prion amplification technique for example); proteins can be extracted from a sample, a sample can be homogenized or sonicated, or combinations thereof.

The term “subject” as used herein can refer to any individual or patient to which the methods described herein can be performed, and specifically from whom a sample can be collected. Generally the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

A sample collected from a subject can be contacted with a host cell comprising one or more vectors described herein. The host cell can be exposed to an excitation light. An emission light signal can be detected. A TDP-43 peptide or aggregate can be detected in the sample, and a TDP-43 related neuropathological disease or condition can be detected in a subject.

For example, emission light signals can be compared to positive or negative controls as described above and/or to a standard curve as described above. In the case of a negative control nothing disturbs the interaction between a TDP-43 peptide linked or fused to a donor fluorescent protein and a TDP-43 peptide linked or fused to an acceptor fluorescent protein and a emission light signal can be detected. In the case of an internal control, no TDP-43 related light can be emitted, an auto-fluorescent signal that can be emitted by a cell can be detected, but no emission light signal can be detected as a result of a transfer of energy from a donor fluorescent protein to an acceptor fluorescent protein. In the case of a negative control exogenous TDP-43 polypeptide fragment or aggregate disturbs the interaction between a TDP-43 peptide linked to a donor fluorescent protein and a TDP-43 peptide linked to an acceptor fluorescent protein; and a lesser or no emission light signal can be detected.

If an emission light signal measured in a sample collected from a subject is equivalent to or greater than a positive control, it can indicate that a sample does not comprise TDP-43 polypeptide fragment or aggregate; and that the subject does not have a TDP-43 related neuropathological disease or condition.

If an emission light signal measured in a sample collected from a subject is less than a positive control, or greater than a negative control, it can indicate that a sample does comprise TDP-43 fragments or aggregates; and that the subject has a TDP-43 related neuropathological disease or condition. Alternatively, if an emission light measured in a sample collected from a subject is greater than or equivalent to a negative control, it can indicate that a sample does comprise TDP-43 polypeptide fragments or aggregates; and that the subject has a TDP-43 related neuropathological disease or condition. Similarly, if an emission light measured in a sample is less than or equivalent to a negative control and greater than an internal control, it can indicate that a sample does comprise TDP-43 polypeptide fragments or aggregates; and that the subject has a TDP-43 related neuropathological disease or condition.

If an emission light measured in a sample is less than an internal control, it can indicate that a test is inconclusive, and no conclusion can be reach regarding the presence or absence of TDP-43 polypeptide fragments or aggregates in a sample; and therefore regarding the detection of a TDP-43 related neuropathological disease or condition in a subject.

The emission light signal can be compared to a standard curve comparing emission light signals obtained in the presence of predetermined amounts of TDP-43 polypeptide peptide. For example, emission light signals measured in the presence of various concentrations of TDP-43 polypeptide fragments can be used to generate a standard curve. If an emission light signal measured in a sample is less than a positive control, but greater than a negative control, it can then be compared to the emission light signal in an standard curve, to estimate the concentration (i.e., the titer) of TDP-43 polypeptide fragments or aggregates present in the sample.

Methods of Identifying a TDP-43 Prion Aggregation Inhibitor

An embodiment provides methods for identifying TDP-43 prion aggregation inhibitors.

Neurodegenerative disease and conditions are generally fatal, with few existing options to slow-down, halt, inhibit or even reverse the accumulation of pathological proteins responsible for the neurodegeneration. Therefore, identifying TDP-43 prion aggregation inhibitors, by assessing if a putative TDP-43 prion aggregation inhibitor can impact the detection of TDP-43 polypeptide fragment or aggregate in a sample (as detailed above), can be used to identify such inhibitors.

A host cell comprising one or more vectors as described herein can be contacted with one or more putative TDP-43 peptide aggregation inhibitors, selected from a library of compounds for example. The host cells can be exposed to an excitation light. An emission light signal can be detected. TDP-43 prion aggregation, or lack thereof can be detected in the sample; and TDP-43 prion aggregation inhibitor can be identified.

For example, an emission light signal measured in the absence of a test compound, or in the presence of a compound known for not being a TDP-43 prion aggregation inhibitor can be used as a positive control. In such case, nothing disturbs the interaction between a TDP-43 peptide linked to a donor fluorescent protein and a TDP-43 peptide linked to an acceptor fluorescent protein; and a emission light signal can be detected. An emission light signal measured in the presence of TDP-43 polypeptide fragments or aggregates, or in the presence of a compounds known for being a TDP-43 prion aggregation inhibitor can be used as a negative control. In such case, TDP-43 polypeptide fragments or aggregates, or a TDP-43 prion aggregation inhibitor can interact with TDP-43 peptide linked to either a donorfluorescent protein or an acceptor fluorescent protein, thereby disturbing the interaction between a TDP-43 peptide linked to a donor fluorescent protein and a TDP-43 peptide linked to an acceptor fluorescent protein, and generating a distance between them. An emission light signal measured in cells that do not express a TDP-43 polypeptide fragment linked to a donor fluorescent protein nor a TDP-43 polypeptide fragment linked to an acceptor fluorescent protein (i.e., a cell expressing a TDP-43 polypeptide fragment linked to a donor fluorescent protein only, a cell expressing a TDP-43 polypeptide fragment linked to an acceptor fluorescent protein only, or a cell not expressing any TDP-43 polypeptide fragment) can be used as an internal control, to evaluate any auto-fluorescent signal that can be emitted by a cell. In such case, no emission light signal can be detected as a result of a transfer of energy from a donor fluorescent protein to an acceptor fluorescent protein; an nothing but cell autofluorescence can be detected.

If an emission light signal measured in a sample comprising a test compound is equivalent to or greater than a positive control, it can indicate that a TDP-43 peptide linked to a donor fluorescent protein can interact with a TDP-43 peptide linked to an acceptor fluorescent protein in the sample, and that a test compound is not a TDP-43 prion aggregation inhibitor.

If an emission light signal measured in a sample comprising a test compound is equivalent or less than a negative control, or if an emission light signal is greater than a negative control and less than a positive control, it can indicate that a TDP-43 peptide linked to a donor fluorescent protein can't fully interact with a TDP-43 peptide linked to an acceptor fluorescent protein in the sample, and that a test compound is a TDP-43 prion aggregation inhibitor.

If an emission light measured in a sample is less than an internal control, it can indicate that a test is inconclusive, and no conclusion can be reach regarding the presence or absence of TDP-43 fragments or aggregates in a sample; and therefore regarding the status of a compound as a TDP-43 prion aggregation inhibitor.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined below to provide additional guidance to the practitioner regarding the description of the compositions and methods. As used in the description herein and throughout the claims that follow, the meaning of “a”, “an”, and “the” includes plural reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

Any single term, single element, single phrase, group of terms, group of phrases, or group of elements described herein can be each be specifically excluded from the claims.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### EXAMPLES

### Example 1. Preparation of TDP-43 Constructs

The accumulation and spread of protein aggregates in neurodegenerative diseases, such as Alzheimer's Disease and Parkinson's Disease, can be explained using a model based on mechanisms seen in prion diseases. This model proposes that a natively folded protein undergoes a conformational change and becomes capable of forming pathogenic aggregates. These aggregates then act as templates for self-replication as they spread from cell to cell. Ultimately, this process leads to cellular dysfunction and neurodegeneration.

Transactive Response Element (TAR) DNA-binding protein-43 (TDP-43) is a protein involved in transcription and splicing, spanning 414 amino acids containing two RNA binding regions, and a glycine-rich C-terminal domain. TDP-43 forms reversible aggregates found in RNA-rich inclusions such as stress granules, but also forms irreversible aggregates found in amyotrophic lateral sclerosis (ALS) and frontotemporal lobar degeneration (FTLD-TDP). In both cases, TDP-43 escapes the nucleus to form insoluble aggregates in the cytoplasm.

As no biosensor exists for TDP-43, TDP-43 constructs that allow for the generation of biosensor cell lines that can reliably detect TDP-43 aggregation upon the application of misfolded TDP-43 were produced.

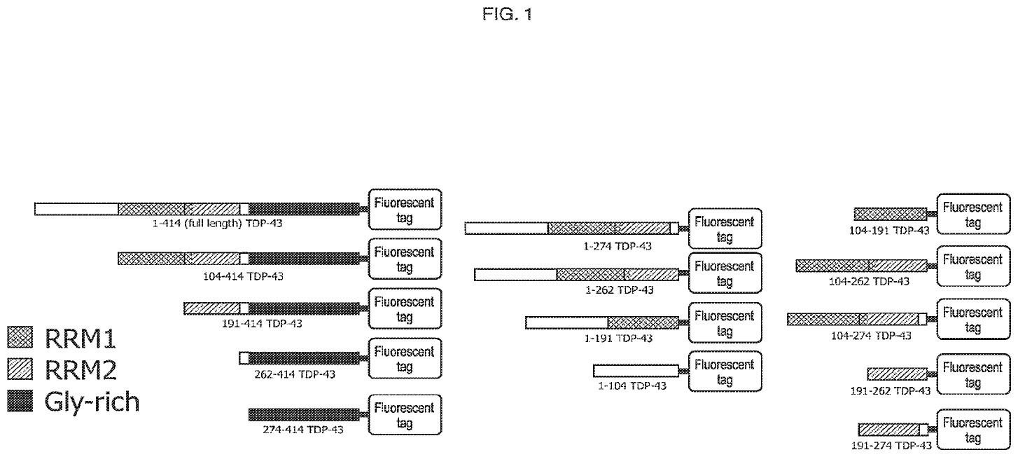

As illustrated in FIG. 1, several TDP-43 constructs were designed based on several key protein domains: RNA recognition motifs I and II (RRM1, aa 104-200 and RRM2, aa191-262) and the glycine-rich C-terminal region (aa 274-414). To enhance the Kozak sequence, an alanine (GCG) was added prior to the start of each protein truncation. A total of 14 TDP-43 constructs were generated and cloned into an FM5 plasmid that had been replaced with a CMV promoter and an mClover3 or mRuby3 fluorescent tag.

### Example 2. Preparation of TDP-43 Biosensor Cell Lines

To generate TDP-43 biosensor cell lines, HEK293T cells were transfected with lentivirus specific to each truncation described in Example 1, to generate cells lines expressing both an mClover3 and man Ruby3 version of each truncation. Cells expressed both fluorescent proteins so that aggregation of TDP-43 could later be measured and quantified via FRET signal on a flow cytometer.

TDP-43 biosensors were first treated with 5 ug of clarified brain homogenate from 14 different patient samples (12 FTLD-TDP Type A, 1 FTLD-TDP Type C, and 1 control) to test cellular response to misfolded TDP-43. Brain homogenate was mixed with a transfection reagent (Lipofectamine 2000) before being added to cells. Cell lines were evaluated in a yes (+) or no (−) manner based on visual examination of inclusion appearance 72 hours after addition of homogenate.

As illustrated in Table 2, different truncations showed different TDP-43 localization (nuclear or cytoplasmic). Responses were determined by response to at least one brain (determined by appearance of inclusions). Only two cells lines showed a response detectable by eye: 262-414 and 274-414.

TABLE 2
Summary of Tested TDP-43 Biosensor Cell Lines.

| Cell line | Localization | FRET Response |
|---|---|---|
| 1-414 | Nuclear; toxic | − |
| 104-414 | cytoplasmic | − |
| 191-414 | cytoplasmic | − |
| 262-414 | cytoplasmic | + |
| 274-414 | cytoplasmic | + |
| 1-104 | nuclear | − |
| 1-191 | nuclear | − |
| 1-262 | nuclear | − |
| 1-274 | nuclear | − |
| 104-191 | cytoplasmic | − |
| 104-262 | cytoplasmic | − |
| 104-274 | cytoplasmic | − |
| 191-262 | cytoplasmic | − |
| 191-274 | cytoplasmic | − |

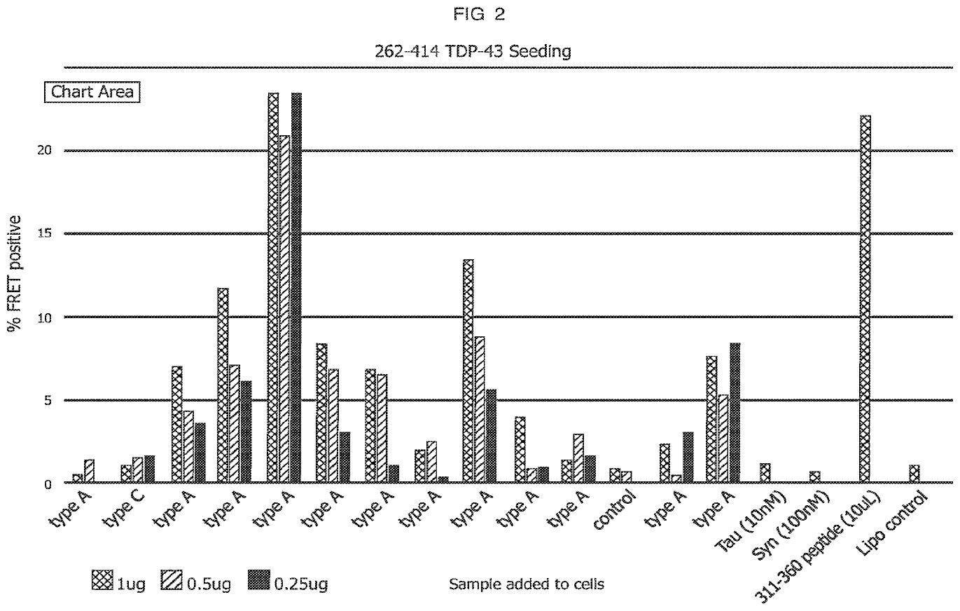

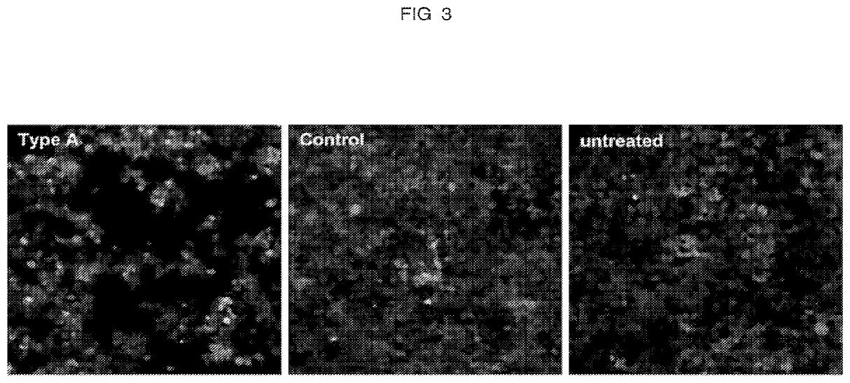

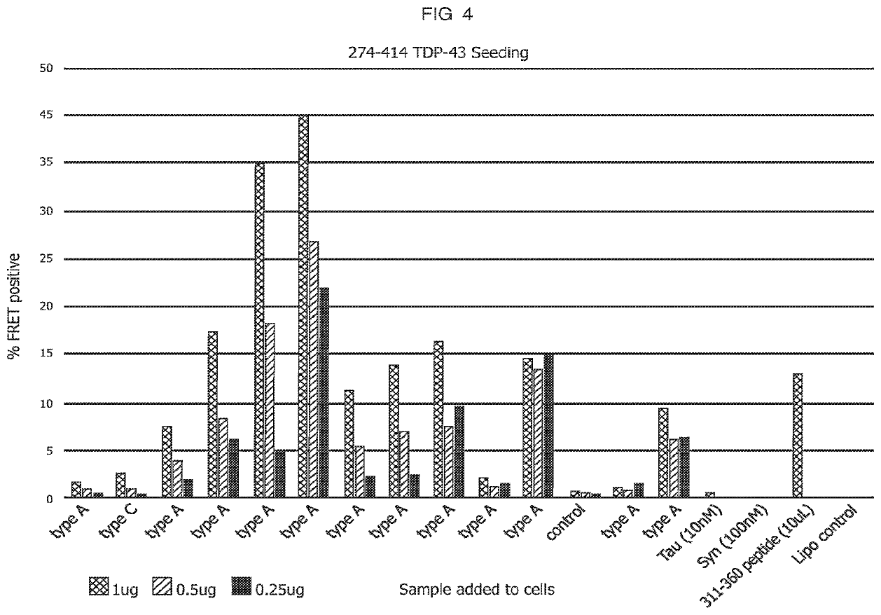

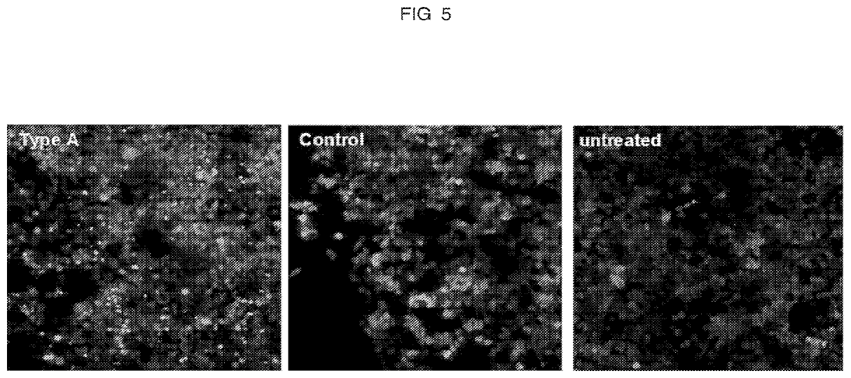

Of the 14 cell lines tested, two lines responded upon addition of brain samples: 262-414 TDP-43 and 274-414 TDP-43. These two cell lines were sorted to make monoclonal populations and further tested. A dose titration of brain samples (1 ug, 0.5 ug, 0.25 ug) were added to test cell sensitivity. To test for specificity to TDP-43 material, cells were also treated with recombinant tau fibrils (10 nM), recombinant alpha-synuclein fibrils (100 nM), and a synthesized TDP-43 peptide (aa311-360). Cells were imaged and ran on a flow cytometer to quantify aggregation 72 hours after addition of samples, as illustrated in FIGS. 2 and 4, showing the quantification of 262-414, and 274-414 respectively FRET signals after sample addition. 72 hours after sample addition, the cells were fixed and ran on a flow cytometer to measure FRET. Brain samples are represented A-N(control brain is L). As further illustrated in FIGS. 3 and 5, it was shown that using 3 ug of homogenized brain, both 274-414 and 262-414 cells can detect TDP-43 aggregates.

TDP-43 has been daunting as a target, given the difficulty to study the full-length protein in cultured cells (which is toxic). Through empirical experimentation, it has been determined that two constructs (aa262-414 and aa274-414) in contradistinction to multiple others functioned as potent biosensors, and monoclonal cell lines in which they function have been generated.

The first monoclonal biosensor cell line was infected by two independent lentivirus vectors, to induce the stable expression of a first expression cassette encoding a TDP-43 (262-414) fragment fused to a linker sequence along with mClover3 as a fluorescent protein (see SEQ ID NO:1), and of a second expression cassette encoding a TDP-43 (262-414) fragment fused to a linker sequence along with mRuby3 as a fluorescent protein (see SEQ ID NO:2)

The second monoclonal biosensor cell line was infected by two independent lentivirus vectors, to induce the stable expression of a first expression cassette encoding a TDP-43 (274-414) fragment fused to a linker sequence along with mClover3 as a fluorescent protein (see SEQ ID NO:5), and of a second expression cassette encoding a TDP-43 (274-414) fragment fused to a linker sequence along with mRuby3 as a fluorescent protein (see SEQ ID NO:6)

In those two cell lines, the same cell expresses the same fragment of TDP-43 polypeptide fragment fused to a linker sequence and to fluorescent protein and each fragment is carried by a different construct. However, the expression cassettes can also be combined into a single plasmid or vector, and the TDP-43 fragments could be non-identical, as long as they can aggregate with one another.

The two cell lines that responded to TDP-43 material both contained the glycine-rich C-terminal region. Despite the only difference between the two lines being the 12-amino acid stretch between 262-274, different brains responded differently to the different cell lines, even amongst the same diseases type. This potential suggests heterogeneity amongst type A brains, although a greater number of brains of other types should be tested (only one type C brain was tested). These experiments identified a TDP-43 truncation that selectively responds to TDP-43.

What is claimed is:

1. An expression cassette comprising one or more polynucleotides encoding a polypeptide at least 95% identical to a sequence as set forth in SEQ ID NO:4 or 8.

2. The expression cassette of claim 1, wherein the one or more polynucleotides comprise a sequence at least 95% identical to a sequence as set forth in SEQ ID NO:3 or SEQ ID NO:7.

3. The expression cassette of claim 1, further comprising:
(a) a polynucleotide encoding a promoter; and
(b) a polynucleotide encoding a fluorescent protein.

4. The expression cassette of claim 3, further comprising a polynucleotide encoding a linker sequence.

5. The expression cassette of claim 4, wherein the linker sequence is present between the polynucleotide encoding the fluorescent protein and the polynucleotide encoding a polypeptide at least 95% identical to a sequence as set forth in SEQ NO:4 or SEQ ID NO:8.

6. The expression cassette of claim 3, wherein the fluorescent protein is a fluorescent donor protein or a fluorescent acceptor protein of a proximity detection protein pair.

7. A host cell comprising the expression cassette of claim 1.

8. A vector comprising the expression cassette of claim 1.

9. The vector of claim 8, comprising a polynucleotide at least 95% identical to a sequence as set forth in SEQ ID NO:1, SEQ ID NO:2, SEQ ID NO:5, or SEQ ID NO:6.

10. A host cell comprising:
(a) a first vector comprising a polynucleotide encoding a first TDP-43 polypeptide fragment and a polynucleotide encoding a fluorescent donor protein, and a second vector comprising a polynucleotide encoding a second TDP-43 polypeptide fragment and a polynucleotide encoding a fluorescent acceptor protein; or
(b) a vector comprising a first polynucleotide encoding a first TDP-43 polypeptide fragment a polynucleotide encoding a fluorescent donor protein, and a second polynucleotide encoding a second TDP-43 polypeptide fragment and a polynucleotide encoding a fluorescent acceptor protein.

11. The host cell of claim 10, wherein the first TDP-43 polypeptide fragment and the second TDP-43 polypeptide fragment are identical TDP-43 polypeptide fragments, or TDP-43 polypeptide fragments of different lengths that co-assemble.

12. The host cell of claim 10, wherein the polynucleotide encoding the first TDP-43 polypeptide fragment comprises a sequence at least 95% identical to a sequence as set forth in SEQ ID NO:3 and the polynucleotide encoding the second TDP-43 polypeptide fragment comprises a sequence at least 95% identical to a sequence as set forth in SEQ ID NO:3.

13. The host cell of claim 10, wherein the polynucleotide encoding the first TDP-43 polypeptide fragment comprises a sequence at least 95% identical to a sequence as set forth in SEQ ID NO:7 and the polynucleotide encoding the second TDP-43 polypeptide fragment comprises a sequence at least 95% identical to a sequence as set forth in SEQ ID NO:7.

14. The host cell of claim 10, wherein the fluorescent donor protein and the fluorescent acceptor protein are members of a proximity detection protein pair.

15. The host cell of claim 14, wherein the proximity detection protein pair is mClover3/mRuby3, EBFP2/mEGFP, ECFP/EYFP, CeruleanNenus, MiCy/mKO, CyPet/YPet, EGFP/mCherry, Venus/mCherry, Venus/tdTomato, or Venus/mPlum.

16. The host cell of claim 10, wherein the first vector comprises a polynucleotide at least 95% identical to a sequence as set forth in SEQ ID NO:1 and the second vector comprises a polynucleotide at least 95% identical to a sequence as set forth in SEQ ID NO:2.

17. The host cell of claim 10, wherein the first vector comprises a polynucleotide at least 95% identical to a sequence as set forth in SEQ ID NO:5 and the second vector comprises a polynucleotide at least 95% identical to a sequence as set forth in SEQ ID NO:6.

18. A method of measuring a titer of or of detecting a TDP-43 peptide or aggregate in a sample comprising:
(a) contacting the sample with the host cell of claim 10;
(b) exposing the host cell to an excitation light; and
(c) detecting an emission light signal,
thereby detecting a TDP-43 peptide or aggregate in the sample.

19. A method of detecting amyotrophic lateral sclerosis (ALS); frontotemporal dementia (FTD), or a neuropathological disease or condition linked to TDP-43 in a subject comprising:
(a) contacting a sample with the host cell of claim 10;
(b) exposing the host cell to an excitation light; and
(c) detecting an emission light signal,
thereby detecting TDP-43 peptide in the sample.

20. The method of claim 18 or 19, wherein the sample is a biological fluid, a tissue sample, or an aggregated material amplified in vitro therefrom.

21. The method of claim 18, or 19, wherein detecting an emission light signal indicates that the sample does not comprise TDP-43 peptide or aggregate.

22. The method of claim 18, or 19, wherein a lack of an emission light signal indicates that the sample comprises TDP-43 peptide or aggregate.

23. A method of identifying a TDP-43 prion aggregation inhibitor comprising:
(a) contacting the host cell of claim 10 with a putative TDP-43 prion aggregation inhibitor;
(b) exposing the host cell to an excitation light;
(c) detecting an emission light signal, and
(d) identifying a TDP-43 prion aggregation inhibitor,
wherein a TDP-43 prion aggregation inhibitor interacts with TDP-43 peptide.

24. The method of claim 23, wherein detecting an emission light signal indicates that the putative TDP-43 peptide aggregation inhibitor does not inhibit TDP-43 peptide aggregation.

25. The method of claim 23, wherein a lack of an emission light signal indicates that the putative TDP-43 peptide aggregation inhibitor inhibits TDP-43 peptide aggregation.

26. The method of claim 18, 19, or 23, wherein the TDP-43 peptide is a pathological TDP-43 prion.

27. The method of claim 18, 19, or 23, wherein detecting an emission light signal is by immunofluorescent microscopy or by flow cytometry.


SEQUENCE LISTING (28 sequences)

SEQ ID NO: 1 - DNA, 9789 bp, Artificial Sequence
  FM5_CMV_TDP-43(262-414)-mRuby3
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccgccaccatggcgaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgggttctgctggctccgctgctggatccggcgaattcgtgtctaagggcgaagagctgatcaaggaaaatatgcgtatgaaggtggtcatggaaggttcggtcaacggccaccaattcaaatgcacaggtgaaggagaaggcagaccgtacgagggaactcaaaccatgaggatcaaagtcatcgagggaggacccctgccatttgcctttgacattcttgccacgtcgttcatgtatggcagccgtacttttatcaagtacccggccgacatccctgatttctttaaacagtcctttcctgagggttttacttgggaaagagttacgagatacgaagatggtggagtcgtcaccgtcacgcaggacaccagccttgaggatggcgagctcgtctacaacgtcaaggtcagaggggtaaactttccctccaatggtcccgtgatgcagaagaagaccaagggttgggagcctaatacagagatgatgtatccagcagatggtggtctgagaggatacactgacatcgcactgaaagttgatggtggtggccatctgcactgcaacttcgtgacaacttacaggtcaaaaaagaccgtcgggaacatcaagatgcccggtgtccatgccgttgatcaccgcctggaaaggatcgaggagagtgacaatgaaacctacgtagtgcaacgcgaagtggcagttgccaaatacagcaaccttggtggtggcatggacgagctgtacaagtaaggcgcgccgggatcgtacaagtaaagcggccgcgactctagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 2 - DNA, 9795 bp, Artificial Sequence
  FM5_CMV_TDP-43(262-414)-mClover3
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccgccaccatggcgaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgggttctgctggctccgctgctggatccggcgaattcgtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaaggcgcgccgggatcgtacaagtaaagcggccgcgactctagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 3 - DNA, 456 bp, Artificial Sequence
  TDP-43 262-414
aagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatg

SEQ ID NO: 4 - protein, 152 aa, Artificial Sequence
  TDP-43 262-414
KHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 5 - DNA, 9753 bp, Artificial Sequence
  FM5_CMV_TDP-43(274-414)-mRuby3
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccgccaccatggcgagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgggttctgctggctccgctgctggatccggcgaattcgtgtctaagggcgaagagctgatcaaggaaaatatgcgtatgaaggtggtcatggaaggttcggtcaacggccaccaattcaaatgcacaggtgaaggagaaggcagaccgtacgagggaactcaaaccatgaggatcaaagtcatcgagggaggacccctgccatttgcctttgacattcttgccacgtcgttcatgtatggcagccgtacttttatcaagtacccggccgacatccctgatttctttaaacagtcctttcctgagggttttacttgggaaagagttacgagatacgaagatggtggagtcgtcaccgtcacgcaggacaccagccttgaggatggcgagctcgtctacaacgtcaaggtcagaggggtaaactttccctccaatggtcccgtgatgcagaagaagaccaagggttgggagcctaatacagagatgatgtatccagcagatggtggtctgagaggatacactgacatcgcactgaaagttgatggtggtggccatctgcactgcaacttcgtgacaacttacaggtcaaaaaagaccgtcgggaacatcaagatgcccggtgtccatgccgttgatcaccgcctggaaaggatcgaggagagtgacaatgaaacctacgtagtgcaacgcgaagtggcagttgccaaatacagcaaccttggtggtggcatggacgagctgtacaagtaaggcgcgccgggatcgtacaagtaaagcggccgcgactctagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 6 - DNA, 9759 bp, Artificial Sequence
  FM5_CMV_TDP-43(274-414)-mClover3
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgccgccaccatggcgagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgggttctgctggctccgctgctggatccggcgaattcgtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaaggcgcgccgggatcgtacaagtaaagcggccgcgactctagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 7 - DNA, 420 bp, Artificial Sequence
  TDP-43 (274-414)
agatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatg

SEQ ID NO: 8 - protein, 140 aa, Artificial Sequence
  TDP-43 (274-414)
RFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 9 - protein, 15 aa, Artificial Sequence
  flexible linker to increase stability/folding and to improve biological activity (GGGGS)3
GGGGSGGGGSGGGGS

SEQ ID NO: 10 - protein, 8 aa, Artificial Sequence
  flexible linker to increase stability/folding (Gly)8
GGGGGGGG

SEQ ID NO: 11 - protein, 6 aa, Artificial Sequence
  flexible linker to increase stability/folding (Gly)6
GGGGGG

SEQ ID NO: 12 - protein, 15 aa, Artificial Sequence
  rigid linker to increase stability/folding (EAAAK)3 or (EAAAK)n (n+1-3)
EAAAKEAAAKEAAAK

SEQ ID NO: 13 - protein, 46 aa, Artificial Sequence
  rigid linker to increase expression, improve biological activity, or to alter PK A(EAAAK)4ALEA(EAAAK)4A
AEAAAKEAAAKEAAAKEAAAKALEAEAAAKEAAAKEAAAKEAAAKA

SEQ ID NO: 14 - protein, 5 aa, Artificial Sequence
  flexible linker to improve biological activity
GGGGS

SEQ ID NO: 15 - protein, 5 aa, Artificial Sequence
  rigid linker to improve biological activity
PAPAP

SEQ ID NO: 16 - protein, 12 aa, Artificial Sequence
  rigid linker to improve biological activity
AEAAAKEAAAKA

SEQ ID NO: 17 - protein, 10 aa, Artificial Sequence
  flexible linker to improve biological activity (GGGGS)n (n=1, 2, 4)
GGGGSGGGGS

SEQ ID NO: 18 - protein, 10 aa, Artificial Sequence
  rigid linker to improve biological activity (Ala-Pro)n (10 - 34 aa)
APAPAPAPAP

SEQ ID NO: 19 - protein, 17 aa, Artificial Sequence
  cleavable linker to enable targeting
VSQTSKLTRAETVFPDV

SEQ ID NO: 20 - protein, 6 aa, Artificial Sequence
  cleavable linker to enable targeting
PLGLWA

SEQ ID NO: 21 - protein, 6 aa, Artificial Sequence
  cleavable linker to enable targeting
RVLAEA

SEQ ID NO: 22 - protein, 10 aa, Artificial Sequence
  cleavable linker to enable targeting
EDVVCCSMSY

SEQ ID NO: 23 - protein, 8 aa, Artificial Sequence
  cleavable linker to enable targeting
GGIEGRGS

SEQ ID NO: 24 - protein, 10 aa, Artificial Sequence
  cleavable linker to enable targeting
TRHRQPRGWE

SEQ ID NO: 25 - protein, 10 aa, Artificial Sequence
  cleavable linker to enable targeting
AGNRVRRSVG

SEQ ID NO: 26 - protein, 9 aa, Artificial Sequence
  cleavable linker to enable targeting
RRRRRRRRR

SEQ ID NO: 27 - protein, 4 aa, Artificial Sequence
  cleavable linker to enable targeting
GFLG

SEQ ID NO: 28 - protein, 414 aa, Artificial Sequence
  TDP-43 full-length protein (1-414)
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM